(19) United States
(12) Patent Application Publication
(10) Pub. No.: US 2026/0317468 A1
(43) Pub. Date: Oct. 15, 2026

(54) TRANSDERMAL DRUG DELIVERY PATCH COMPRISING A COPOLYMER HYDROGEL AND METHOD OF PREPARING THE SAME

(71) Applicant: RECENTCARE BIOMEDICAL INC., Taipei City (TW)

(72) Inventors: Hung Pin TSENG, Taipei (TW); Pei-Chun SHIH, Taipei (TW)

(73) Assignee: RECENTCARE BIOMEDICAL INC., Taipei City (TW)

(21) Appl. No.: 19/644,012

(22) Filed: Apr. 10, 2026

Related U.S. Application Data
(60) Provisional application No. 63/787,379, filed on Apr. 11, 2025.

(51) Int. Cl. C08F 228/02; A61K 9/70; A61K 31/352; A61K 31/405; C08F 2/10; C08F 2/48; C08F 20/26; C08K 5/20

(52) U.S. Cl. CPC C08F 228/02; A61K 9/703; A61K 31/352; A61K 31/405; C08F 2/10; C08F 2/48; C08F 20/26; C08K 5/20

(57) ABSTRACT

A transdermal drug delivery patch and a method of preparing the same are disclosed. The patch comprises a pre-cured hydrogel layer and an active ingredient layer disposed on the hydrogel layer. The hydrogel layer features a crosslinked copolymer network comprising polymerized units of an acrylic sulfonate monomer and an acrylic amide monomer. To preserve the stability of sensitive active pharmaceutical ingredients (APIs), the hydrogel layer is crosslinked via ultraviolet (UV) irradiation prior to drug loading. An active ingredient solution containing an API and a co-solvent is subsequently coated onto the pre-cured hydrogel matrix. The removal of excess water from the matrix induces the API into a highly stable, supersaturated state. This specific copolymer network and post-cure coating method synergistically enhance transdermal drug flux and release kinetics while preventing UV-induced API degradation.

### CROSS REFERENCE TO RELATED APPLICATIONS

This non-provisional application claims the benefit under 35 U.S.C. § 119 (e) to U.S. Provisional Application No. 63/787,379, filed on Apr. 11, 2025, which is hereby expressly incorporated by reference into the present application.

### FIELD OF THE INVENTION

The present invention relates generally to the field of pharmaceutical formulations and targeted drug delivery. More specifically, the present invention relates to a hydrogel-based transdermal drug delivery system (TDDS) comprising a specifically formulated crosslinked copolymer network. The present invention further relates to methods of manufacturing said transdermal patch, particularly methods utilizing a post-polymerization drug loading process designed to preserve the stability of active pharmaceutical ingredients while driving the active ingredients into a highly bioavailable, supersaturated state to enhance transdermal flux and release kinetics.

### BACKGROUND OF THE INVENTION

Transdermal drug delivery systems (TDDS) provide a highly effective alternative to conventional administration routes, such as oral delivery or intravenous injection. By facilitating the direct absorption of active pharmaceutical ingredients (APIs) through the skin, TDDS bypasses first-pass hepatic metabolism, reduces gastrointestinal adverse effects, and improves patient compliance through non-invasive administration. However, the clinical efficacy of TDDS is inherently limited by the barrier properties of the stratum corneum. To achieve therapeutic efficacy, a transdermal patch must provide a sufficient concentration gradient to drive the API across the skin, a property largely dictated by the solubility and release kinetics of the API within the patch matrix.

Hydrogels are frequently utilized as carrier matrices in TDDS due to their biocompatibility and favorable swelling properties. Conventional manufacturing of UV-crosslinked hydrogel patches typically involves an “in situ” loading approach, where the API is mixed directly into a prepolymer or monomer solution prior to ultraviolet (UV) irradiation. A major limitation of this approach is that UV exposure and the resulting free radicals can degrade, photo-oxidize, or otherwise inactivate structurally sensitive APIs. This severely restricts the spectrum of drug candidates compatible with UV-cured hydrogel formulations.

To circumvent UV-induced degradation, post-cure drug loading—such as coating an API solution onto a pre-formed hydrogel—has been explored. However, conventional hydrogel matrices (such as standard sodium acrylate-based networks) often exhibit poor or sluggish drug release profiles when loaded post-polymerization. Because the polymer network is already crosslinked and heavily hydrated, applying an active ingredient solution often results in suboptimal drug partitioning and slow, continuous low-level release kinetics that fail to provide the initial burst or sustained flux required for therapeutic efficacy.

Furthermore, maximizing transdermal flux often requires maintaining the API in a highly concentrated, or ideally, a supersaturated state. Achieving and maintaining supersaturation within a hydrogel matrix is thermodynamically complex and highly unpredictable. In conventional hydrogels, forcing an API into high concentrations typically leads to rapid recrystallization, which drastically reduces bioavailability. The specific interactions between crosslinked polymer chains, retained water molecules, and applied co-solvent solutions are difficult to predict, making the rational design of a matrix capable of inherently driving and stabilizing a supersaturated API state a persistent challenge in the art.

Given the foregoing limitations, there remains a critical and unmet need for an advanced TDDS formulation that physically protects sensitive APIs from UV degradation while simultaneously possessing a unique structural matrix capable of interacting with applied solvents to unexpectedly drive APIs into a supersaturated, high-release state, thereby overcoming the slow-release limitations of conventional post-cured hydrogels.

### BRIEF SUMMARY OF THE INVENTION

The present invention provides, in one aspect, a transdermal drug delivery patch, comprising: a hydrogel layer comprising a crosslinked copolymer network, wherein the crosslinked copolymer network comprises polymerized units of an acrylic sulfonate monomer and an acrylic amide monomer; and an active ingredient layer disposed on at least one surface of the hydrogel layer, wherein the active ingredient layer comprises an active pharmaceutical ingredient and a co-solvent.

According to certain embodiments of the invention, the acrylic sulfonate monomer is selected from the group consisting of 2-acrylamido-2-methylpropane sulfonic acid (AMPS) and 3-sulfopropyl acrylate potassium salt, and wherein the acrylic amide monomer is selected from the group consisting of N,N-dimethylacrylamide (NDMA) and N-isopropylacrylamide (NIPA).

According to certain embodiments of the invention, a weight ratio of the polymerized units of the acrylic sulfonate monomer to the polymerized units of the acrylic amide monomer in the crosslinked copolymer network is between 0.5:1 and 8:1, more preferably between 1:1 and 5:1, and most preferably 2:1.

According to certain embodiments of the invention, the crosslinked copolymer network further comprises a crosslinker selected from the group consisting of poly(ethylene glycol) diacrylate (PEGDA) and N,N′-methylenebisacrylamide (NMBA).

According to certain embodiments of the invention, the co-solvent is selected from the group consisting of glycerol, diethylene glycol monoethyl ether, polyethylene glycol 400 (PEG400), 1-methyl-2-pyrrolidinone (NMP), polyoxyethylene (20) sorbitan monolaurate, polyoxyethylene (20) sorbitan monooleate, sorbitan monooleate, propylene glycol, 2-(2-ethoxyethoxy) ethanol, and combinations thereof.

According to certain embodiments of the invention, the active pharmaceutical ingredient is selected from the group consisting of indomethacin and Rose Bengal.

According to certain embodiments of the invention, a thickness of the hydrogel layer ranges from 0.25 mm to 10 mm. Preferably, the thickness of the hydrogel layer ranges from 0.25 mm to 0.35 mm. In one example, the thickness of the hydrogel layer is about 0.3 mm.

According to certain embodiments of the invention, a thickness of the active ingredient layer ranges from 0.05 mm to 5 mm. Preferably, the thickness of the active ingredient layer ranges from 0.15 mm to 2 mm.

It is to be understood that both the foregoing general description and the following detailed description are exemplary and explanatory only and are not restrictive of the invention.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

FIG. 1 is a line graph illustrating the cumulative release percentage of Indomethacin over time, comparing the release kinetics of the inventive post-cured coated hydrogel patches against comparative pre-mixed hydrogel matrices.

FIG. 2 is a line graph illustrating the cumulative release percentage of Rose Bengal over time, comparing the release kinetics of the inventive post-cured coated hydrogel patches against comparative pre-mixed hydrogel matrices.

FIG. 3 is a line graph illustrating the cumulative release percentage of Rose Bengal over time, comparing the release kinetics of inventive hydrogel patches comprising specific acrylic sulfonate and acrylic amide monomer combinations against a comparative conventional hydrogel patch.

FIG. 4 is a line graph illustrating the cumulative release percentage of Indomethacin over time, comparing the release kinetics of inventive hydrogel patches comprising specific acrylic sulfonate and acrylic amide monomer combinations against a comparative conventional hydrogel patch.

### DETAILED DESCRIPTION OF THE INVENTION

It is to be understood that this disclosure is not limited to the particular methods, compositions, and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless otherwise defined herein, scientific and technical terms used herein have the meanings that are commonly understood by those of ordinary skill in the art to which this invention belongs.

### I. Definitions

As used herein, the indefinite articles “a” and “an” and the definite article “the” are intended to include both the singular and the plural, unless the context in which they are used clearly indicates otherwise. The term “comprising” is intended to be open-ended, meaning that the composition or method includes the recited elements, but does not exclude others.

As used herein, the term “transdermal drug delivery system” (TDDS) refers to any device, patch, film, or formulation designed to deliver a therapeutically effective amount of an active pharmaceutical ingredient (API) across the skin and into the systemic circulation or targeted local tissues.

As used herein, the term “hydrogel” or “hydrogel layer” refers to a three-dimensional, crosslinked polymeric network capable of absorbing and retaining a significant amount of water or biological fluids without dissolving. In the context of the present invention, the hydrogel serves as the primary structural carrier or matrix for the TDDS.

As used herein, the term “crosslinker” refers to a chemical compound that chemically binds two or more polymer chains or monomers together by covalent or ionic bonds to form a three-dimensional network.

As used herein, the term “photo-initiator” refers to a molecule that absorbs light energy (such as ultraviolet or visible light) and subsequently decomposes into reactive species, such as free radicals, cations, or anions, which initiate the polymerization and crosslinking of monomers.

As used herein, the term “co-solvent” or “co-solvent solution” refers to any solvent or mixture of solvents used in combination with water or other primary solvents to enhance the solubility, stability, or skin permeation of an active pharmaceutical ingredient.

As used herein, the term “supersaturated state” or “supersaturated solution” refers to a thermodynamic state in which a solvent contains a higher concentration of a dissolved solute (e.g., an active pharmaceutical ingredient) than it could hold under normal equilibrium conditions at a given temperature and pressure, without the solute precipitating or recrystallizing.

As used herein, the term “coating” refers to the process of applying a formulation containing an active ingredient onto at least one surface of a pre-formed or pre-cured hydrogel matrix. The coating may be applied as a liquid, semi-solid, gel, or aerosolized mist.

As used herein, the term “evaporation” refers to the process by which a volatile liquid (e.g., water or a volatile co-solvent) dissipates from the hydrogel or the coated layer into the surrounding environment, which may be accelerated by environmental controls such as heat, vacuum, or airflow.

### II. The Hydrogel Matrix Composition

The present invention features a hydrogel layer serving as a carrier matrix for use in a TDDS. The hydrogel layer comprises a crosslinked copolymer network formed from at least two distinct classes of monomers: an acrylic sulfonate monomer and an acrylic amide monomer.

Acrylic Sulfonate Monomer: The acrylic sulfonate monomer imparts highly hydrophilic properties and ionic characteristics to the polymer network. In some embodiments, the acrylic sulfonate monomer is present in an amount ranging from about 5% to about 40% by weight of the pre-polymerized hydrogel solution. Specific examples of suitable acrylic sulfonate monomers include, but are not limited to, 2-acrylamido-2-methylpropane sulfonic acid (AMPS), 3-sulfopropyl acrylate potassium salt, sodium styrene sulfonate, 2-sulfoethyl methacrylate, 3-sulfopropyl methacrylate, and salts or derivatives thereof.

Acrylic Amide Monomer: The acrylic amide monomer provides structural integrity and specific hydrogen-bonding capabilities within the network. In some embodiments, the acrylic amide monomer is present in an amount ranging from about 5% to about 40% by weight of the hydrogel solution. Specific examples of suitable acrylic amide monomers include, but are not limited to, N,N-dimethylacrylamide (NDMA), N-isopropylacrylamide (NIPA), acrylamide, methacrylamide, N,N-diethylacrylamide, N-hydroxymethyl acrylamide, and combinations thereof.

Monomer Ratios: The synergistic effect of the present invention is highly dependent on the interaction between these two monomers. In some embodiments, the weight percentage of the acrylic sulfonate monomer is 0.5 to 8 times the weight percentage of the acrylic amide monomer, more preferably 1 to 5 times, and most preferably 2 times. This specific ratio optimizes the formation of “crystal water” domains within the matrix, which is critical for driving the active ingredient into a supersaturated state upon loading.

Crosslinkers: To form the insoluble three-dimensional network, a crosslinker is incorporated. The crosslinker may be present in an amount ranging from about 0.1% to about 5% by weight of the hydrogel solution. Suitable crosslinkers include, but are not limited to, poly(ethylene glycol) diacrylate (PEGDA), N,N′-methylenebisacrylamide (NMBA), ethylene glycol dimethacrylate (EGDMA), N,N′-bis(acryloyl) cystamine, divinylbenzene, and combinations thereof.

Photo-initiators: The polymerization process is driven by a photo-initiator, typically present in an amount ranging from about 0.01% to about 0.5% by weight of the hydrogel solution. Suitable photo-initiators include, but are not limited to, hydroxycyclohexyl phenyl ketone (e.g., Irgacure 184), 2-hydroxy-2-methyl-1-phenyl-propan-1-one (e.g., Darocur 1173), 4,4′-azo-bis(4-cyanopentanoic acid), 2-hydroxy-1-[4-(2-hydroxyethoxy)phenyl]-2-methyl-1-propanone (e.g., Irgacure 2959), lithium phenyl-2,4,6-trimethylbenzoylphosphinate (LAP), and Eosin Y.

### III. The Active Ingredient Layer and Supersaturation

The transdermal patch further comprises an active ingredient layer disposed on the hydrogel layer. This layer comprises an active pharmaceutical ingredient (API) and a co-solvent.

Active Pharmaceutical Ingredients: The present invention is uniquely suited for delivering APIs that are structurally sensitive to UV degradation or require a high concentration gradient to permeate the stratum corneum. Suitable APIs include, but are not limited to: non-steroidal anti-inflammatory drugs (NSAIDs) such as indomethacin, diclofenac, ibuprofen, and ketoprofen; diagnostic agents or dyes such as Rose Bengal; hormones such as estrogen, progesterone, and testosterone; analgesics such as fentanyl and buprenorphine; and anti-hypertensives such as clonidine.

Co-Solvents: The co-solvent facilitates the initial dissolution of the API and interacts with the hydrogel matrix. Suitable co-solvents include, but are not limited to, glycerol, diethylene glycol monoethyl ether (e.g., Transcutol-P), polyethylene glycol 400 (PEG 400), 1-methyl-2-pyrrolidinone (NMP), polysorbates (e.g., polyoxyethylene (20) sorbitan monolaurate, polyoxyethylene (20) sorbitan monooleate), sorbitan monooleate, propylene glycol, 2-(2-ethoxyethoxy) ethanol, methanol, ethanol, isopropyl myristate, and combinations thereof.

Mechanism of Supersaturation: The present invention is based at least in part on the unexpected finding that a hydrogel comprising the specific combination of an acrylic sulfonate monomer and an acrylic amide monomer effectively retains “crystal water” bound within its matrix. When an active ingredient solution (comprising the API and co-solvent) is coated onto this pre-cured matrix, the unique properties of the copolymer network force the release of excess bulk water. As this excess water is removed via evaporation, the API is prevented from crystallizing and is instead driven into a stable, supersaturated state within the co-solvent and polymer microenvironments. This supersaturation creates a massive thermodynamic driving force, resulting in a fast-release kinetic profile and enhanced molecular flux (J) across the skin, as governed by Fick's law of diffusion.

### IV. Manufacturing Methods and Physical Characteristics

Method of Preparation: The present invention provides a method of preparing the TDDS that preserves API integrity. The process generally comprises: (a) mixing the monomers, crosslinker, and photo-initiator to form a pre-polymer solution; (b) exposing the solution to UV light (e.g., 365 nm at 700 mW/cm² for 5 to 15 seconds) to form a pre-cured hydrogel layer; (c) independently mixing an API with a co-solvent to form an active ingredient solution; (d) coating the active ingredient solution onto the pre-cured hydrogel; and (e) evaporating excess water to induce supersaturation.

Coating Techniques: The coating step avoids incorporating the API prior to UV exposure. Suitable coating methods include spray coating (e.g., ultrasonic spray coating), spin coating, dip coating, doctor blade coating, puddle dispensing, slot-die coating, flow-through coating, aerosol printing, and inkjet printing.

Evaporation Techniques: The evaporation step may be carried out under ambient conditions, or accelerated via heating, forced air drying, vacuum drying, or freeze-drying (lyophilization).

Dimensions: The physical dimensions of the patch may be tailored to the required therapeutic dosage. In some embodiments, the thickness of the hydrogel layer ranges from about 0.1 mm to about 10 mm, preferably from 0.25 mm to 0.35 mm, and most preferably about 0.3 mm. The active ingredient layer coated thereon may have an effective thickness ranging from about 0.05 mm to about 5 mm, preferably from 0.15 mm to 2 mm.

### EXAMPLES

The following examples are provided to illustrate certain embodiments of the invention and are not intended to limit the scope of the invention as defined by the appended claims.

### Example 1: Evaluation of Drug Release Kinetics Based on Drug Loading Methods (Indomethacin)

### 1. Materials and Methods

### 1.1.1 Preparation of Pre-Mixed Hydrogel Matrix (Comparative)

1. A monomer solution was formed by mixing 15 mg of NDMA and 35.6 mg of AMPS in 35.6 mL of glycerol.
2. A hydrogel solution was obtained by mixing 4.2 mg of PEGDA (crosslinker), 0.2 mg of hydroxycyclohexyl phenyl ketone (photo-initiator), and 50 mg of a co-solvent solution with the monomer solution for 30 minutes.
3. 1 mg of Indomethacin (IND) was mixed with solvents and the hydrogel solution obtained in step 2.
4. The mixture was poured into a donor chamber of a Franz cell.

The comparative hydrogel matrix was obtained by exposing the mixture to 365 nm UV light at an energy level of 700 mW/cm² for 5 seconds.

### 1.1.2 Preparation of Post-Cured Coated Patch (Inventive)

1. A monomer solution was formed by mixing 15 mg of NDMA and 35.6 mg of AMPS in 35.6 mL of glycerol.
2. A hydrogel solution was obtained by mixing 4.2 mg of PEGDA, 0.2 mg of hydroxycyclohexyl phenyl ketone, and 50 mg of a co-solvent solution with the monomer solution for 30 minutes.
3. The hydrogel solution was poured into a donor chamber of a Franz cell.
4. The pre-cured hydrogel patch was obtained by exposing the solution to 365 nm UV light at an energy level of 700 mW/cm² for 5 seconds.
5. An active ingredient solution was obtained by mixing 1 mg of IND with methanol or Transcutol-P.
6. The active ingredient solution was coated onto the pre-cured hydrogel.
7. Excess water was removed by evaporation or heating to obtain the final patch, thereby inducing the active ingredient into a supersaturated state within the hydrogel.

### 1.2 Franz Cell Assay

An artificial skin sheet was utilized to determine the release profile of IND from the hydrogels. The receptor chamber was filled with PBS (pH 7.4) and stirred for 30 minutes by a magnet at 32° C. The hydrogel was clamped between the donor cap and the receptor chamber. Samples were pipetted 5 to 10 times, and 100 μL samples were collected at 30 minutes, 1 hour, 2 hours, 4 hours, and 8 hours. After each collection, 100 μL of fresh PBS was injected into the receptor chamber.

### 2. Results

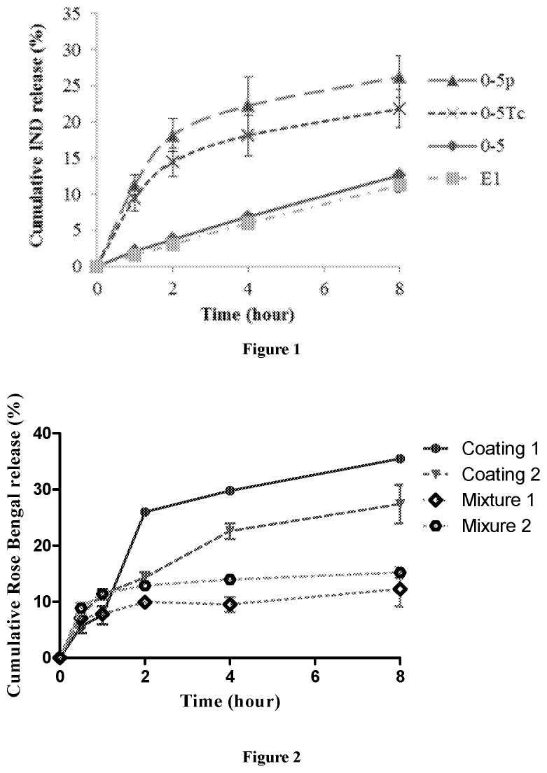

As depicted in FIG. 1 the comparative pre-mixed hydrogels (E1 and 0-5) exhibited a slow and steady release kinetic. In contrast, the inventive post-cured coated hydrogels (0-5Tc and 0-5P) exhibited significantly faster release kinetics. Specifically, the coated patches demonstrated an unexpected initial burst release of IND within the first two hours, driven by the supersaturated state of the API, followed by a transition to a sustained release phase.

### Example 2: Evaluation of Drug Release Kinetics Based on Drug Loading Methods (Rose Bengal)

### 1. Materials and Methods

### 1.1.1 Preparation of Pre-Mixed Hydrogel Matrix (Comparative)

1. A monomer solution was formed by mixing 100 mg of NDMA and 550 mg of AMPS in 250 mL of glycerol.
2. A hydrogel solution was obtained by mixing 30 mg of PEGDA, 5 mg of hydroxycyclohexyl phenyl ketone, and 500 mg of a co-solvent solution with the monomer solution for 30 minutes.
3. 75 μg of Rose Bengal was mixed with Transcutol-P or ultrapure water and the hydrogel solution.
4. 1 g of the mixture was poured into a donor chamber of a Franz cell.
5. The comparative matrix was obtained by exposing the mixture to 365 nm UV light (700 mW/cm²) for 15 seconds.

### 1.1.2 Preparation of Post-Cured Coated Patch (Inventive)

1. A monomer solution was formed by mixing 100 mg of NDMA and 550 mg of AMPS in 250 mL of glycerol.
2. A hydrogel solution was obtained by mixing 30 mg of PEGDA, 5 mg of hydroxycyclohexyl phenyl ketone, and 500 mg of a co-solvent solution with the monomer solution for 30 minutes.
3. 1 g of the hydrogel solution was poured into a donor chamber of a Franz cell.
4. The pre-cured hydrogel patch was obtained by exposing the solution to 365 nm UV light (700 mW/cm²) for 15 seconds.
5. An active ingredient solution was obtained by mixing 50 μg of Rose Bengal with Transcutol-P or ultrapure water.
6. The active ingredient solution was coated onto the pre-cured hydrogel.
7. Excess water was removed by evaporation or heating to induce a supersaturated state of the active ingredient within the hydrogel.

### 1.2 Franz Cell Assay

The assay was conducted under the same conditions as Example 1, except 200 μL samples were collected at 30 minutes, 1, 2, 4, and 8 hours.

### 2. Results

As shown in FIG. 2 and summarized in Table 1, the comparative pre-mixed hydrogels (Mixture 1 and Mixture 2) exhibited a slow initial release of Rose Bengal, reaching cumulative release percentages of only 9.90% and 12.87% at two hours, respectively. Conversely, the inventive coated hydrogels (Coating 1 and Coating 2) exhibited a highly robust release profile within the first four hours, achieving cumulative release percentages of 29.79% and 22.58%, respectively. This data demonstrates that the post-cure coating method applied to the specific monomeric matrix successfully overcomes the slow-release limitations inherent to conventional in-situ mixing.

TABLE 1
Cumulative Release Percentage of Rose Bengal (%). Coating
1 compares to Mixture 1; Coating 2 compares to Mixture 2.

| mean ± SD | Coating 1 | Coating 2 | Mixture 1 | Mixture 2 |
|---|---|---|---|---|
| 0.5 | 5.625 ± 1.199 | 7.66 ± 0.7515 | 7.043 ± 0.6019 | 8.842 ± 0.9927 |
| 1 | 7.589 ± 1.64 | 11.31 ± 0.9050 | 7.781 ± 0.5424 | 11.37 ± 0.3366 |
| 2 | 25.98 ± 0.6629 | 14.3 ± 0.9574 | 9.909 ± 0.7487 | 12.87 ± 0.3799 |
| | ***P = 0.0003 | | | |
| 4 | 29.79 ± 0.4368 | 22.58 ± 1.386 | 9.507 ± 1.393 | 13.95 ± 0.3873 |
| | **P = 0.0023 | *P = 0.0133 | | |
| 8 | 35.47 ± 0.443 | 27.38 ± 3.432 | 12.26 ± 3.092 | 15.18 ± 0.955 |
| | **P = 0.0088 | *P = 0.0378 | | |

### Example 3

### 1. Materials and Methods

### 1.1.1 Preparation of Inventive Hydrogels (Tests 1, 2, and 3)

Pre-cured hydrogel patches were prepared utilizing the specific acrylic sulfonate and acrylic amide monomer combinations of the present invention.
Test 1:200 mg NIPA and 350 mg 3-sulfopropyl acrylate potassium salt.
Test 2:200 mg NDMA and 350 mg 3-sulfopropyl acrylate potassium salt.
Test 3:200 mg NDMA and 350 mg AMPS.

For each test, the monomers were mixed in 250 mL of glycerol. A hydrogel solution was formed by adding 50 mg PEGDA, 5 mg hydroxycyclohexyl phenyl ketone, and 530 mg co-solvent solution. 500 mg of the solution was poured into a Franz cell and UV-cured (365 nm, 700 mW/cm², 5 seconds). Subsequently, 50 μg of Rose Bengal in ultrapure water was coated onto each patch, and excess water was removed via evaporation to achieve supersaturation.

### 1.1.2 Preparation of Conventional Hydrogel (Test 4—Comparative)

1. A monomer solution was formed by mixing 100 mg of NIPA, 5 mg of sodium acrylate (SA), 2 mg of N,N′-methylenebisacrylamide (BIS) crosslinker, and 1000 mg of ultrapure water.
2. A hydrogel solution was obtained by mixing 12 mg of ammonium persulfate and 6 mg of TEMED.
3. Within 10 seconds of adding TEMED, 350 mL of the solution was poured into the Franz cell donor chamber to cure.
4. 50 μg of Rose Bengal in ultrapure water was coated onto the conventional hydrogel, and excess water was removed via evaporation.

### 1.2 Franz Cell Assay

The assay was conducted under the same conditions as Example 1, except 200 μL samples were collected at 30 minutes, 1, 2, 3, and 4 hours.

### 2. Results

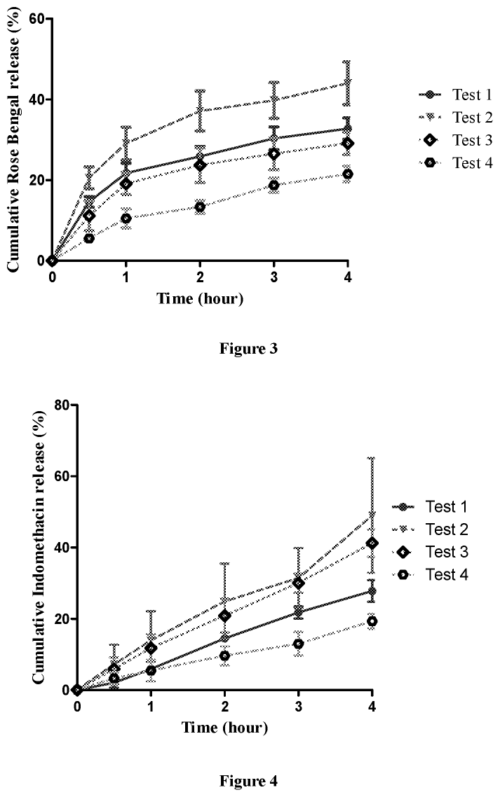

As shown in FIG. 3 and Table 2, the comparative conventional hydrogel (Test 4) exhibited a substantially constant, low release rate, achieving only 5.53% release at 0.5 hours. In unexpected contrast, the inventive hydrogels utilizing the specific combination of an acrylic sulfonate monomer and an acrylic amide monomer (Tests 1, 2, and 3) exhibited an immediate and robust burst release. At 0.5 hours, Tests 1 and 2 achieved cumulative releases of 14.58% and 20.55%, respectively—an approximately threefold increase over the conventional matrix. This demonstrates the unique synergy between the claimed copolymer matrix and the post-cure coating method.

TABLE 2
Cumulative Release Percentage of Rose Bengal (%).
Test 1, Test 2, and Test 3 compared to Test 4.

| mean ± SD | Test 1 | Test 2 | Test 3 | Test 4 |
|---|---|---|---|---|
| 0.5 | 14.58 ± 1.328 | 20.55 ± 2.721 | 11.15 ± 3.725 | 5.539 ± 1.328 |
| | **P = 0.0054 | *P = 0.0172 | | |
| 1 | 21.72 ± 2.447 | 29.07 ± 4.030 | 19.09 ± 2.712 | 10.5 ± 2.36 |
| | *P = 0.0229 | *P = 0.0142 | *P = 0.048 | |
| 2 | 25.86 ± 2.457 | 37.1 ± 4.985 | 23.67 ± 4.299 | 13.33 ± 1.597 |
| | *P = 0.0117 | *P = 0.0224 | | |
| 3 | 30.33 ± 2.851 | 39.73 ± 4.448 | 26.5 ± 3.958 | 18.70 ± 1.784 |
| | *P = 0.0203 | *P = 0.0241 | | |
| 4 | 32.75 ± 2.67 | 43.98 ± 5.31 | 29.12 ± 2.792 | 21.5 ± 1.922 |
| | *P = 0.0209 | *P = 0.0288 | | |

### Example 4: Comparative Analysis of Hydrogel Matrix Compositions (Indomethacin)

### 1. Materials and Methods

### 1.1.1 Preparation of Inventive Hydrogels (Tests 1, 2, and 3)

The hydrogel matrices for Tests 1, 2, and 3 were prepared identically to Example 3. Instead of Rose Bengal, an active ingredient solution comprising 200 μg of Indomethacin mixed with methanol was coated onto each pre-cured patch, followed by evaporation to achieve supersaturation.

### 1.1.2 Preparation of Conventional Hydrogel (Test 4—Comparative)

The conventional hydrogel matrix (NIPA, SA, and BIS) was prepared identically to Example 3. The patch was subsequently coated with the same 200 μg Indomethacin/methanol solution, followed by evaporation.

### 1.2 Franz Cell Assay

The assay was conducted under the same conditions as Example 3.

### 2. Results

As shown in FIG. 4 and Table 3, while all groups demonstrated sustained release over time, the comparative conventional hydrogel (Test 4) suffered from a significantly slower release profile. The inventive hydrogels (Tests 1, 2, and 3) exhibited superior release kinetics. For example, Test 3 achieved cumulative releases of 30.00% and 41.23% at the 3-hour and 4-hour time points, representing a significant increase over the 12.98% and 19.29% respective releases observed in Test 4. These results further confirm that the structural copolymer composition of the present invention uniquely facilitates enhanced transdermal flux when paired with a coated API solution.

TABLE 3
Cumulative Release Percentage of Indomethacin (%).
Test 1, Test 2, and Test 3 compared to Test 4.

| mean ± SD | Test 1 | Test 2 | Test 3 | Test 4 |
|---|---|---|---|---|
| 0.5 | 2.037 ± 1.401 | 7.116 ± 5.642 | 5.92 ± 3.214 | 3.29 ± 1.608 |
| 1 | 5.893 ± 0.9072 | 13.94 ± 8.186 | 11.75 ± 3.844 | 5.438 ± 2.971 |
| 2 | 14.5 ± 0.5739 | 9.601 ± 2.633 | 20.87 ± 4.753 | 9.601 ± 2.633 |
| 3 | 21.79 ± 1.751 | 31.41 ± 8.429 | 30.00 ± 2.677 | 12.98 ± 3.325 |
| | | | * P = 0.0141 | |
| 4 | 27.81 ± 3.074 | 48.97 ± 16.09 | 41.23 ± 3.85 | 19.29 ± 2.059 |
| | | | **P = 0.0076 | |

We claim:

1. A transdermal drug delivery patch, comprising: a hydrogel layer comprising a crosslinked copolymer network, wherein the crosslinked copolymer network comprises polymerized units of an acrylic sulfonate monomer and an acrylic amide monomer; and an active ingredient layer disposed on at least one surface of the hydrogel layer, wherein the active ingredient layer comprises an active pharmaceutical ingredient and a co-solvent.

2. The transdermal drug delivery patch of claim 1, wherein the acrylic sulfonate monomer is selected from the group consisting of 2-acrylamido-2-methylpropane sulfonic acid (AMPS) and 3-sulfopropyl acrylate potassium salt, and wherein the acrylic amide monomer is selected from the group consisting of N,N-dimethylacrylamide (NDMA) and N-isopropylacrylamide (NIPA).

3. The transdermal drug delivery patch of claim 1, wherein a weight ratio of the polymerized units of the acrylic sulfonate monomer to the polymerized units of the acrylic amide monomer in the crosslinked copolymer network is between 0.5:1 and 8:1.

4. The transdermal drug delivery patch of claim 3, wherein a weight ratio of the polymerized units of the acrylic sulfonate monomer to the polymerized units of the acrylic amide monomer in the crosslinked copolymer network is between 1:1 and 5:1.

5. The transdermal drug delivery patch of claim 4, wherein a weight ratio of the polymerized units of the acrylic sulfonate monomer to the polymerized units of the acrylic amide monomer in the crosslinked copolymer network is 2:1.

6. The transdermal drug delivery patch of claim 1, wherein the crosslinked copolymer network further comprises a crosslinker selected from the group consisting of poly(ethylene glycol) diacrylate (PEGDA) and N,N′-methylenebisacrylamide (NMBA).

7. The transdermal drug delivery patch of claim 1, wherein the co-solvent is selected from the group consisting of glycerol, diethylene glycol monoethyl ether, polyethylene glycol 400 (PEG400), 1-methyl-2-pyrrolidinone (NMP), polyoxyethylene (20) sorbitan monolaurate, polyoxyethylene (20) sorbitan monooleate, sorbitan monooleate, propylene glycol, 2-(2-ethoxyethoxy) ethanol, and combinations thereof.

8. The transdermal drug delivery patch of claim 1, wherein the active pharmaceutical ingredient is selected from the group consisting of indomethacin and Rose Bengal.

9. The transdermal drug delivery patch of claim 1, wherein the hydrogel layer has a thickness ranging from 0.25 mm to 10 mm.

10. The transdermal drug delivery patch of claim 1, wherein the active ingredient layer has a thickness ranging from 0.05 mm to 5 mm.

11. A method for preparing a transdermal drug delivery patch, comprising the steps of: (a) providing a pre-cured hydrogel layer comprising a crosslinked copolymer network, wherein the crosslinked copolymer network is formed by ultraviolet (UV) curing a hydrogel solution comprising an acrylic sulfonate monomer and an acrylic amide monomer; (b) mixing an active pharmaceutical ingredient with a co-solvent to obtain an active ingredient solution; (c) coating the active ingredient solution obtained in step (b) onto the pre-cured hydrogel layer; and (d) removing excess water by evaporation or heating to induce the active pharmaceutical ingredient into a supersaturated state, thereby obtaining the transdermal drug delivery patch.

12. The method of claim 11, wherein step (a) comprises: (i) mixing the acrylic sulfonate monomer, the acrylic amide monomer, a crosslinker, and a photo-initiator to obtain the hydrogel solution; and (ii) exposing the hydrogel solution to UV light to polymerize the monomers into the crosslinked copolymer network prior to coating the active ingredient solution.

13. The method of claim 12, wherein the hydrogel solution comprises 5% to 40% by weight of the acrylic sulfonate monomer and 5% to 40% by weight of the acrylic amide monomer.

14. The method of claim 12, wherein the photo-initiator is selected from the group consisting of hydroxycyclohexyl phenyl ketone, 2-hydroxy-2-methyl-1-phenyl-propan-1-one, 4,4′-azo-bis(4-cyanopentanoic acid), and 2-hydroxy-1-[4-(2-hydroxyethoxy)phenyl]-2-methyl-1-propanone.
